(19) United States
(12) Patent Application Publication
(10) Pub. No.: US 2012/0253197 A1
(43) Pub. Date: Oct. 4, 2012

(54) SYSTEMS AND METHODS FOR FLUSHING BUBBLES FROM A CATHETER OF AN INTRAVASCULAR ULTRASOUND IMAGING SYSTEM

(75) Inventor: Alain Sadaka, San Jose, CA (US)

(73) Assignee: Boston Scientific SciMed, Inc., Maple Grove, MN (US)

(21) Appl. No.: 13/431,483

(22) Filed: Mar. 27, 2012

Related U.S. Application Data
(60) Provisional application No. 61/469,254, filed on Mar. 30, 2011.

(51) Int. Cl. A61B 8/12

(52) U.S. Cl. 600/444; 600/445

(57) ABSTRACT

An intravascular ultrasound imaging system includes a catheter that is insertable into a patient blood vessel. A flushing assembly is in fluid communication with a lumen of the catheter. The flushing assembly flushes air bubbles formed in acoustically-favorable medium in the catheter lumen in response to an event other than a user-initiated flushing prompt. The flushing assembly includes a reservoir containing acoustically-favorable medium for input into the catheter lumen, and a pump coupled to the reservoir. The pump pumps the acoustically-favorable medium from the reservoir to the catheter lumen. A connector is in fluid communication with the reservoir. The connector is coupleable with the catheter lumen. A controller is coupled to the pump. The controller is configured and arranged for controlling operation of the pump.

### CROSS-REFERENCE TO RELATED APPLICATIONS

This application claims the benefit under 35 U.S.C. §119(e) of U.S. Provisional Patent Application Ser. No. 61/469,254 filed on Mar. 30, 2011, which is incorporated herein by reference.

### TECHNICAL FIELD

The present invention is directed to the area of imaging systems that are insertable into a patient and methods of making and using the imaging systems. The present invention is also directed to automated assemblies for flushing bubbles from catheters of the imaging systems, as well as methods of making and using the flushing assemblies, catheters, and imaging systems.

### BACKGROUND

Ultrasound devices insertable into patients have proven diagnostic capabilities for a variety of diseases and disorders. For example, intravascular ultrasound (“IVUS”) imaging systems have been used as an imaging modality for diagnosing blocked blood vessels and providing information to aid medical practitioners in selecting and placing stents and other devices to restore or increase blood flow. IVUS imaging systems have been used to diagnose atheromatous plaque build-up at particular locations within blood vessels. IVUS imaging systems can be used to determine the existence of an intravascular obstruction or stenosis, as well as the nature and degree of the obstruction or stenosis. IVUS imaging systems can be used to visualize segments of a vascular system that may be difficult to visualize using other intravascular imaging techniques, such as angiography, due to, for example, movement (e.g., a beating heart) or obstruction by one or more structures (e.g., one or more blood vessels not desired to be imaged). IVUS imaging systems can be used to monitor or assess ongoing intravascular treatments, such as angiography and stent placement in real (or almost real) time. Moreover, IVUS imaging systems can be used to monitor one or more heart chambers.

IVUS imaging systems have been developed to provide a diagnostic tool for visualizing a variety of diseases or disorders. An IVUS imaging system can include a control module (with a pulse generator, an image processor, and a monitor), a catheter, and one or more transducers disposed in the catheter. The transducer-containing catheter can be positioned in a lumen or cavity within, or in proximity to, a region to be imaged, such as a blood vessel wall or patient tissue in proximity to a blood vessel wall. The pulse generator in the control module generates electrical signals that are delivered to the one or more transducers and transformed to acoustic signals that are transmitted through patient tissue. Reflected signals of the transmitted acoustic signals are absorbed by the one or more transducers and transformed to electric signals. The transformed electric signals are delivered to the image processor and converted to an image displayable on the monitor.

### BRIEF SUMMARY

In one embodiment, an intravascular ultrasound imaging system includes a catheter that is insertable into a patient blood vessel. The catheter has a distal end, a proximal end, a longitudinal axis, and defines a sealable lumen configured and arranged to receive an acoustically-favorable medium. At least one transducer is configured and arranged for insertion into the lumen of the catheter. The at least one transducer is configured and arranged for transmitting acoustic signals, receiving reflected echo signals corresponding to the transmitted acoustic signals, and transforming the received echo signals to electrical signals. A control module is coupled to the at least one transducer. The control module includes a processor and a drive unit. The processor is configured and arranged for processing electrical signals received from the at least one transducer. The drive unit is configured and arranged for rotating the at least one transducer about, and along, the longitudinal axis of the catheter. A flushing assembly is in fluid communication with the catheter lumen. The flushing assembly is configured and arranged for flushing air bubbles formed in the acoustically-favorable medium disposed in the catheter lumen in response to an event other than a user-initiated flushing prompt. The flushing assembly includes a reservoir configured and arranged for containing acoustically-favorable medium for being input to the catheter lumen. A pump is coupled to the reservoir. The pump is configured and arranged for pumping at least some of the acoustically-favorable medium from the reservoir to the catheter lumen. A connector is in fluid communication with the reservoir. The connector is coupleable with the catheter lumen. A controller is coupled to the pump. The controller is configured and arranged for controlling operation of the pump.

In another embodiment, a method for flushing a lumen of a catheter includes providing an intravascular ultrasound (IVUS) imaging system. The IVUS imaging system includes a catheter that is insertable into a patient blood vessel. The catheter has a distal end, a proximal end, a longitudinal axis, and defines a sealable lumen configured and arranged to receive an acoustically-favorable medium. At least one transducer is configured and arranged for insertion into the lumen of the catheter. The at least one transducer is configured and arranged for transmitting acoustic signals, receiving reflected echo signals corresponding to the transmitted acoustic signals, and transforming the received echo signals to electrical signals. A control module is coupled to the at least one transducer. The control module includes a processor and a drive unit. The processor is configured and arranged for processing electrical signals received from the at least one transducer. The drive unit is configured and arranged for rotating the at least one transducer about, and along, the longitudinal axis of the catheter. A flushing assembly is in fluid communication with the catheter lumen. The flushing assembly is configured and arranged for flushing air bubbles formed in the acoustically-favorable medium disposed in the catheter lumen in response to an event other than a user-initiated flushing prompt. The flushing assembly includes a reservoir configured and arranged for containing acoustically-favorable medium for being input to the catheter lumen. A pump is coupled to the reservoir. The pump is configured and arranged for pumping at least some of the acoustically-favorable medium from the reservoir to the catheter lumen. A connector is in fluid communication with the reservoir. The connector is coupleable with the catheter lumen. A controller is coupled to the pump. The controller is configured and arranged for controlling operation of the pump. The processor of the IVUS imaging system is coupled to the controller of the flushing assembly. The controller receives data from the processor of the IVUS imaging system. The data is generated from electrical signals received from the at least one transducer of the IVUS imaging system as the at least one transducer rotates and moves along the lumen of the catheter of the IVUS imaging system. The controller detects an event other than a user-initiated flushing prompt. The controller initiates pumping of an acoustically-favorable medium from the reservoir of the IVUS imaging system to the lumen of the catheter in response to the event.

In yet another embodiment, a computer-readable medium has processor-executable instructions, the processor-executable instructions when installed on a device enable the device to perform actions. The actions include receiving data from a processor of an intravascular ultrasound (IVUS) imaging system. The data is generated from electrical signals received from at least one transducer of the IVUS imaging system as the at least one transducer rotates and moves along a lumen of a catheter of the IVUS imaging system. The actions also include detecting an event other than a user-initiated flushing prompt, and pumping of an acoustically-favorable medium from a reservoir external to the catheter to the lumen of the catheter in response to the event.

In another embodiment, a system for flushing a lumen of a catheter within which a rotatable intravascular ultrasound (IVUS) imager is disposed includes a processor coupleable to the IVUS imager, and a flushing-assembly controller coupleable to the processor. The processor receives data from the IVUS imager. The flushing-assembly controller executes processor-readable instructions that enable actions. The actions include receiving data from a processor of the IVUS imaging system. The data is obtained as the IVUS imager rotates and moves along the lumen of the catheter. The actions also include detecting an event other than a user-initiated flushing prompt, and pumping of an acoustically-favorable medium from a reservoir external to the catheter to the lumen of the catheter in response to the event.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an ultrasound imaging system suitable for insertion into a patient, the ultrasound imaging system including a catheter and a control module, according to the invention;

FIG. 2 is a schematic side view of one embodiment of the catheter of FIG. 1, according to the invention;

FIG. 3 is a schematic longitudinal cross-sectional view of one embodiment of a distal end of the catheter of FIG. 1 with an imaging core disposed in a lumen defined in a sheath, according to the invention;

FIG. 4A is a schematic view of one embodiment of a flushing assembly, the flushing assembly formed as an integral part of the ultrasound imaging system of FIG. 1, according to the invention;

FIG. 4B is a schematic view of one embodiment of the flushing assembly of FIG. 4A, the flushing assembly formed as a stand-alone structure configured and arranged to couple to the ultrasound imaging system of FIG. 1, according to the invention;

FIG. 5A is a schematic view of one embodiment of the flushing assembly of FIG. 4A, according to the invention;

FIG. 5B is a schematic view of another embodiment of the flushing assembly of FIG. 4A, the flushing assembly including tubing coupling a reservoir of the flushing assembly to the catheter of FIG. 1, according to the invention;

FIG. 5C is a schematic view of yet another embodiment of the flushing assembly of FIG. 4A, the flushing assembly including a feedback loop system coupled to the processor of the control module of FIG. 1, according to the invention; and

FIG. 6 is a flow diagram of one exemplary embodiment of a technique for flushing a lumen of a catheter using the flushing assembly of FIG. 4A, according to the invention.

### DETAILED DESCRIPTION

The present invention is directed to the area of imaging systems that are insertable into a patient and methods of making and using the imaging systems. The present invention is also directed to automated assemblies for flushing bubbles from catheters of the imaging systems, as well as methods of making and using the flushing assemblies, catheters, and imaging systems.

The methods, systems, and devices described herein may be embodied in many different forms and should not be construed as limited to the embodiments set forth herein. Accordingly, the methods, systems, and devices described herein may take the form of an entirely hardware embodiment, an entirely software embodiment or an embodiment combining software and hardware aspects. The methods described herein can be performed using any type of computing device, such as a computer, that includes a processor or any combination of computing devices where each device performs at least part of the process.

Suitable computing devices typically include mass memory and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computing device.

Methods of communication between devices or components of a system can include both wired and wireless (e.g., RF, optical, or infrared) communications methods and such methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

Suitable intravascular ultrasound (“IVUS”) systems include, but are not limited to, one or more transducers disposed on a distal end of a catheter configured and arranged for percutaneous insertion into a patient. Examples of IVUS imaging systems with catheters are found in, for example, U.S. Pat. Nos. 7,246,959; 7,306,561; 7,622,853; and 6,945,938; as well as U.S. Patent Application Publication Nos. 2006/0100522; 2006/0106320; 2006/0173350; 2006/0253028; and 2007/0016054; all of which are incorporated herein by reference.

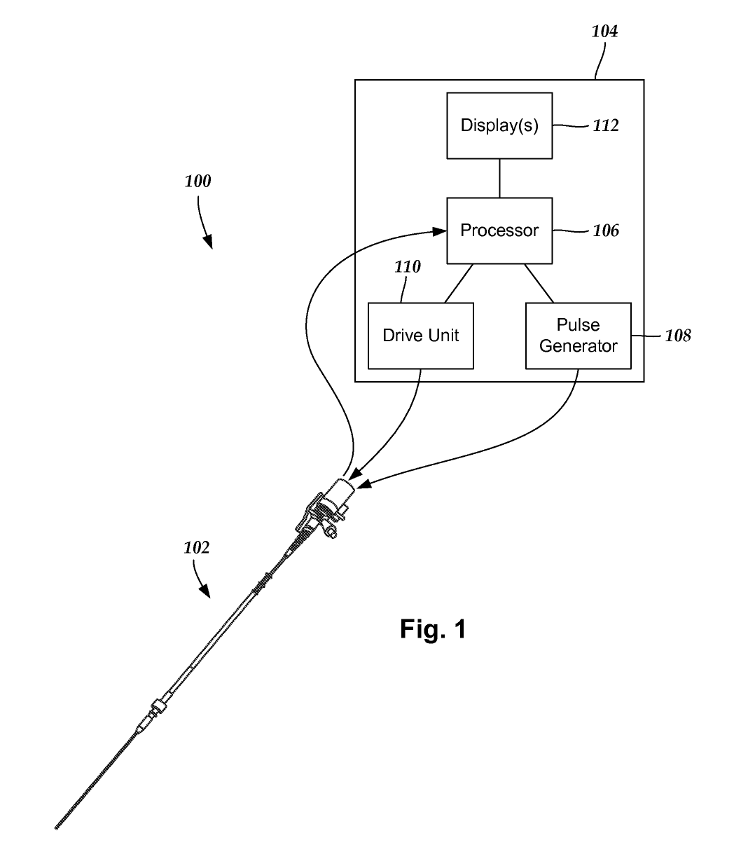

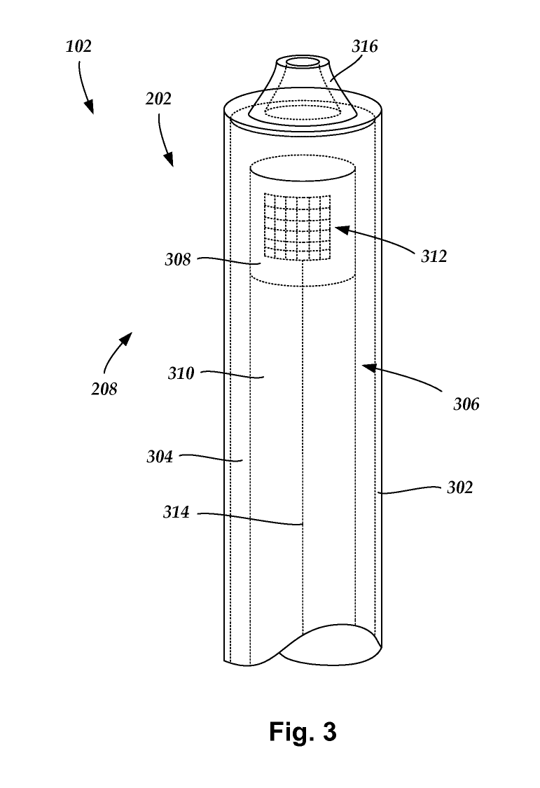

FIG. 1 illustrates schematically one embodiment of an IVUS imaging system 100. The IVUS imaging system 100 includes a catheter 102 that is coupleable to a control module 104. The control module 104 may include, for example, a processor 106, a pulse generator 108, a drive unit 110, and one or more displays 112. In at least some embodiments, the pulse generator 108 forms electric signals that may be input to one or more transducers (312 in FIG. 3) disposed in the catheter 102.

In at least some embodiments, mechanical energy from the drive unit 110 may be used to drive an imaging core (306 in FIG. 3) disposed in the catheter 102. In at least some embodiments, electric signals transmitted from the one or more transducers (312 in FIG. 3) may be input to the processor 106 for processing. In at least some embodiments, the processed electric signals from the one or more transducers (312 in FIG. 3) can be displayed as one or more images on the one or more displays 112. For example, a scan converter can be used to map scan line samples (e.g., radial scan line samples, or the like) to a two-dimensional Cartesian grid to display the one or more images on the one or more displays 112.

In at least some embodiments, the processor 106 may also be used to control the functioning of one or more of the other components of the control module 104. For example, the processor 106 may be used to control at least one of the frequency or duration of the electrical signals transmitted from the pulse generator 108, the rotation rate of the imaging core (306 in FIG. 3) by the drive unit 110, the velocity or length of the pullback of the imaging core (306 in FIG. 3) by the drive unit 110, or one or more properties of one or more images formed on the one or more displays 112.

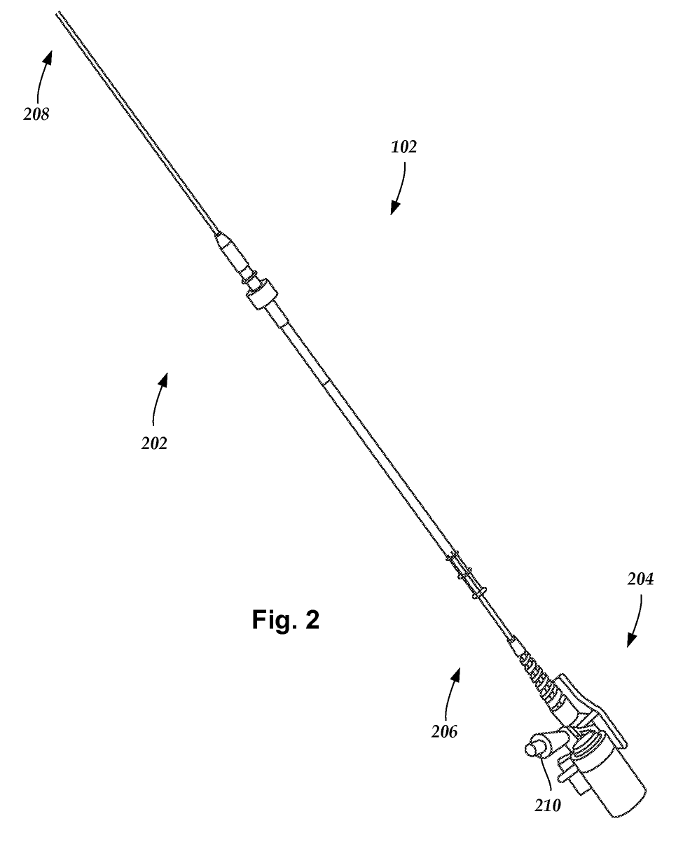

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. Optionally, the catheter 102 may define at least one flush port, such as flush port 210. The flush port 210 may be defined in the hub 204. The hub 204 may be configured and arranged to couple to the control module (104 in FIG. 1). In some embodiments, the elongated member 202 and the hub 204 are formed as a unitary body. In other embodiments, the elongated member 202 and the catheter hub 204 are formed separately and subsequently assembled together.

FIG. 3 is a schematic perspective view of one embodiment of the distal end 208 of the elongated member 202 of the catheter 102. The elongated member 202 includes a sheath 302 with a longitudinal axis 303 and a lumen 304. An imaging core 306 is disposed in the lumen 304. The imaging core 306 includes an imaging device 308 coupled to a distal end of a rotatable driveshaft 310. One or more transducers 312 may be mounted to the imaging device 308 and employed to transmit and receive acoustic signals. The sheath 302 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

In a preferred embodiment (as shown in FIG. 3), an array of transducers 312 are mounted to the imaging device 308. In alternate embodiments, a single transducer may be employed. Any suitable number of transducers 312 can be used. For example, there can be two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used. When a plurality of transducers 312 are employed, the transducers 312 can be configured into any suitable arrangement including, for example, an annular arrangement, a rectangular arrangement, or the like.

The one or more transducers 312 may be formed from one or more known materials capable of transforming applied electrical signals to pressure distortions on the surface of the one or more transducers 312, and vice versa. Examples of suitable materials include piezoelectric ceramic materials, piezocomposite materials, piezoelectric plastics, barium titanates, lead zirconate titanates, lead metaniobates, polyvinylidenefluorides, and the like. Other transducer technologies include composite materials, single-crystal composites, and semiconductor devices (e.g., capacitive micromachined ultrasound transducers (“cMUT”), piezoelectric micromachined ultrasound transducers (“pMUT”), or the like)

The pressure distortions on the surface of the one or more transducers 312 form acoustic signals of a frequency based on the resonant frequencies of the one or more transducers 312. The resonant frequencies of the one or more transducers 312 may be affected by the size, shape, and material used to form the one or more transducers 312. The one or more transducers 312 may be formed in any shape suitable for positioning within the catheter 102 and for propagating acoustic signals of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, microfabrication, and the like.

As an example, each of the one or more transducers 312 may include a layer of piezoelectric material sandwiched between a conductive acoustic lens and a conductive backing material formed from an acoustically absorbent material (e.g., an epoxy substrate with tungsten particles). During operation, the piezoelectric layer may be electrically excited by both the backing material and the acoustic lens to cause the emission of acoustic signals.

The one or more transducers 312 can be used to form a radial cross-sectional image of a surrounding space. Thus, for example, when the one or more transducers 312 are disposed in the catheter 102 and inserted into a blood vessel of a patient, the one more transducers 312 may be used to form an image of the walls of the blood vessel and tissue surrounding the blood vessel.

The imaging core 306 may, optionally, be rotated about the longitudinal axis 303 of the catheter 102. As the imaging core 306 rotates, the one or more transducers 312 emit acoustic signals in different radial directions. When an emitted acoustic signal with sufficient energy encounters one or more medium boundaries, such as one or more tissue boundaries, a portion of the emitted acoustic signal is reflected back to the emitting transducer as an echo signal. Each echo signal that reaches a transducer with sufficient energy to be detected is transformed to an electrical signal in the receiving transducer. The one or more transformed electrical signals are transmitted to the control module (104 in FIG. 1) where the processor 106 processes the electrical-signal characteristics to form a displayable image of the imaged region based, at least in part, on a collection of information from each of the acoustic signals transmitted and the echo signals received. In at least some embodiments, the rotation of the imaging core 306 is driven by the drive unit 110 disposed in the control module (104 in FIG. 1). In alternate embodiments, the one or more transducers 312 are fixed in place and do not rotate. In which case, the driveshaft 310 may, instead, rotate a mirror that reflects acoustic signals to and from the fixed one or more transducers 312.

When the one or more transducers 312 are rotated about the longitudinal axis 303 of the catheter 102 emitting acoustic signals, a plurality of images can be formed that collectively form a radial cross-sectional image of a portion of the region surrounding the one or more transducers 312, such as the walls of a blood vessel of interest and the tissue surrounding the blood vessel. The radial cross-sectional image can, optionally, be displayed on one or more displays 112. The at least one of the imaging core 306 can be either electronically or manually rotated.

The imaging core 306 may also move longitudinally along the blood vessel within which the catheter 102 is inserted so that a plurality of cross-sectional images may be formed along a longitudinal length of the blood vessel. During an imaging procedure the one or more transducers 312 may be retracted (i.e., pulled back) along the longitudinal length of the catheter 102. The catheter 102 can include at least one telescoping section that can be retracted during pullback of the one or more transducers 312. In at least some embodiments, the drive unit 110 drives the pullback of the imaging core 306 within the catheter 102. The drive unit 110 pullback distance of the imaging core can be any suitable distance including, for example, at least 5 cm, 10 cm, 15 cm, 20 cm, 25 cm, or more. The entire catheter 102 can be retracted during an imaging procedure either with or without the imaging core 306 moving longitudinally independently of the catheter 102.

When the imaging core 306 is refracted while rotating, the images may collectively form a continuous spiral shape along a blood vessel. A stepper motor may, optionally, be used to pull back the imaging core 306. The stepper motor can pull back the imaging core 306 a short distance and stop long enough for the one or more transducers 306 to capture an image before pulling back the imaging core 306 another short distance and again capturing another image, and so on, either with or without being rotated.

The quality of an image produced at different depths from the one or more transducers 312 may be affected by one or more factors including, for example, bandwidth, transducer focus, beam pattern, as well as the frequency of the acoustic signal. The frequency of the acoustic signal output from the one or more transducers 312 may also affect the penetration depth of the acoustic signal output from the one or more transducers 312. In general, as the frequency of an acoustic signal is lowered, the depth of the penetration of the acoustic signal within patient tissue increases. In at least some embodiments, the IVUS imaging system 100 operates within a frequency range of 5 MHz to 100 MHz.

One or more conductors 314 can electrically couple the transducers 312 to the control module 104 (see e.g., FIG. 1). In which case, the one or more conductors 314 may extend along a longitudinal length of the rotatable driveshaft 310.

The catheter 102 with one or more transducers 312 mounted to the distal end 208 of the imaging core 308 may be inserted percutaneously into a patient via an accessible blood vessel, such as the femoral artery, femoral vein, or jugular vein, at a site remote from the selected portion of the selected region, such as a blood vessel, to be imaged. The catheter 102 may then be advanced through the blood vessels of the patient to the selected imaging site, such as a portion of a selected blood vessel.

Acoustic signals propagating from the one or more transducers 312 propagate through the lumen 304 of the catheter 102 before passing through the sheath 302 to the region exterior of the catheter 102, such as a blood vessel or a chamber of a heart. Likewise, echo signals reflected back to the one or more transducers 312 from medium boundaries also propagate through the lumen 304 of the catheter 102. Typically, air is not a desirable transmission medium and image quality may, consequently, be reduced when acoustic signals or echo signals are required by catheter design to propagate through air. In the MHz range, acoustic signals may not propagate at all through air.

Accordingly, it is typically advantageous, and in some cases necessary, to purge air bubbles from the lumen 304 surrounding the one or more transducers 312 prior to the performance of an imaging procedure. One technique for purging air surrounding the one or more transducers 312 is to flush the lumen 304 with an acoustically-favorable medium through which acoustic signals more easily propagate than through air Acoustically-favorable media may include one or more solvents such as, for example, water. An acoustically-favorable medium may include one or more solutes mixed with the one or more solvents such as, for example, a saline solution. Optionally, one or more agents may also be added, for example, to decrease the potential advancement of corrosion or microbial growth. An acoustically-favorable medium may include a gel, and the like. In some cases, the acoustically-favorable medium may be input through the main flush port 210. The main flush port 210 can, optionally, be configured to receive a luer connector. In at least some embodiments, the elongated member 202 also defines an output port 316 for outputting one or more gases. The output port 316 may be defined in a distal end of the catheter 102.

When using a conventional IVUS imaging system, a lumen of a catheter may be flushed to remove air at the beginning of an IVUS imaging procedure. Additionally, the lumen of the catheter may also need to be flushed of air one or more additional times during the course of the IVUS imaging procedure. Unfortunately, each flushing of air from the catheter lumen can add to the amount of time it takes for a medical practitioner to perform an IVUS imaging procedure on a patient. Additionally, flushing may involve manually attaching one or more accessory items to the catheter (e.g., a syringe, an extension tube, or the like or combinations thereof). Accordingly, manually flushing the catheter may introduce the potential for human error, as well as the need for using one or more accessories, which may get lost or break, or fail during use, or the like.

As herein described, an automated flushing assembly (“flushing assembly”) is configured and arranged to perform a flushing procedure that flushes air from the catheter. The flushing assembly flushes the catheter upon the occurrence of an event other than a user-initiated flushing prompt. Such events may include, for example, a detectable degradation in image quality, an initiation of an imaging procedure, a passing of a predetermined amount of time, or the like. The flushing assembly 402 may additionally be configured and arranged to perform a flushing procedure in response a user-initiated flushing prompt. Optionally, the flushing assembly 402 may also include a feedback system that makes adjustments to one or more flushing settings in response to received signals.

The flushing assembly may reduce the amount of time to perform a flushing procedure, as well as reducing the exposure to human error and inconsistency, by automating the flushing of the catheter lumen. Reducing the amount of time to perform the flushing procedure may, likewise, reduce the amount of time to perform the imaging procedure. Additionally, providing an automated flushing assembly may reduce complexity of the imaging procedure by eliminating one or more accessories to perform a conventional, manual flushing procedure.

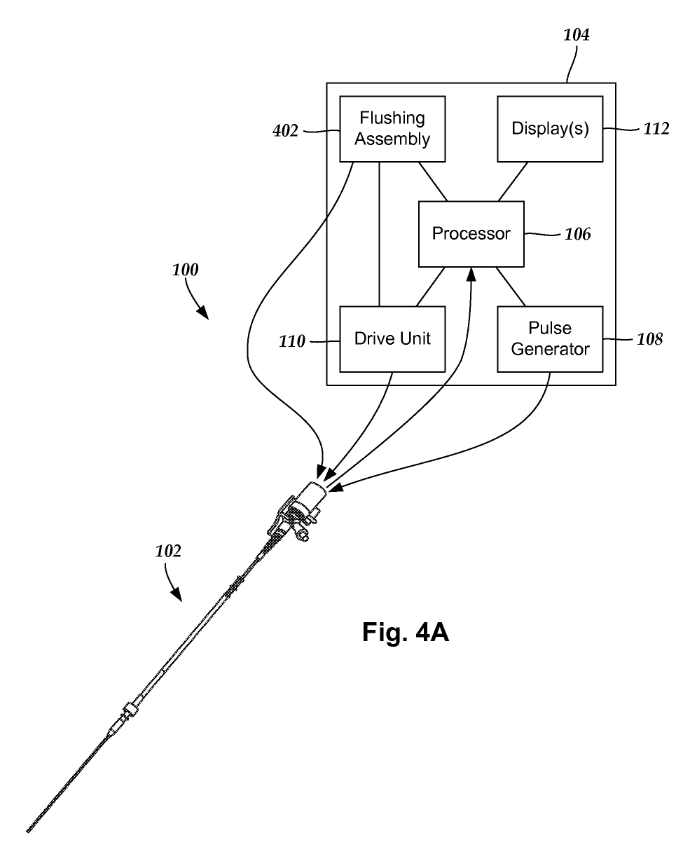

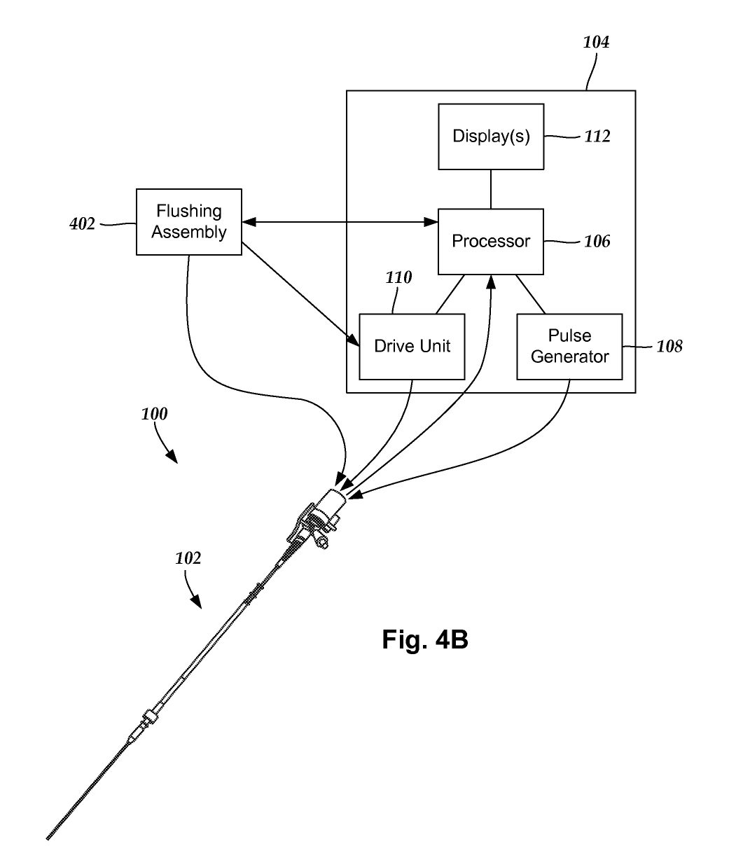

The flushing assembly is disposed external to the catheter and, preferably, disposed external to the patient during a flushing procedure. In some cases, the flushing assembly can be formed as part of the IVUS imaging system 100. FIG. 4A is a schematic view of one embodiment of a flushing assembly 402 disposed within the IVUS imaging system 100. In other cases, the flushing assembly 402 can be formed as a separate device that can be coupled to the IVUS imaging system 100. FIG. 4B is a schematic view of one embodiment of the flushing assembly 402 coupled to the IVUS imaging system 100.

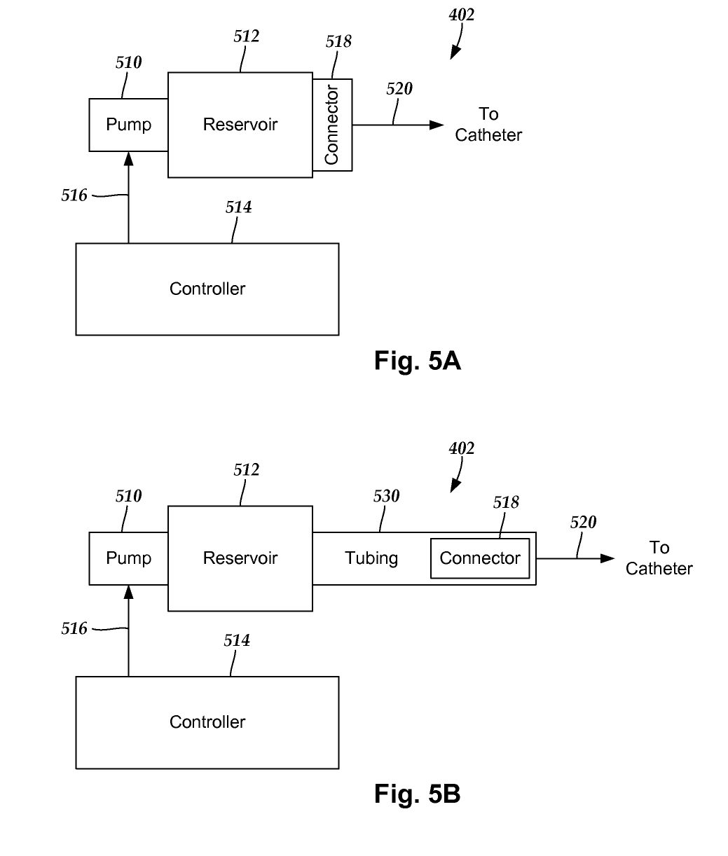

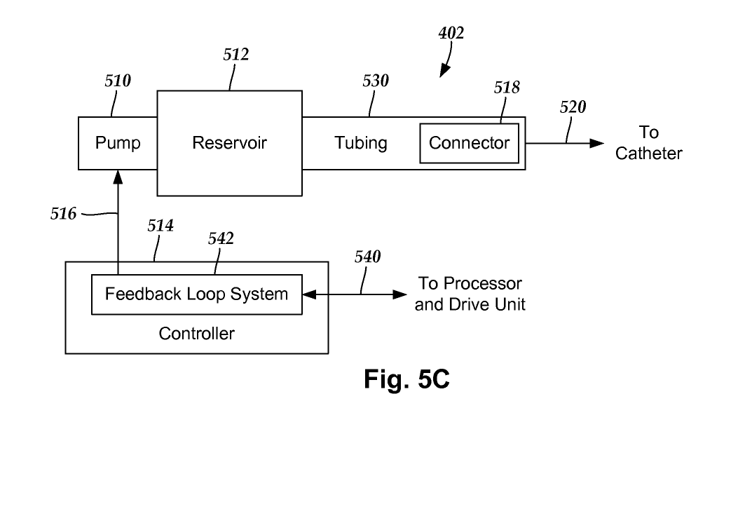

FIG. 5A is a schematic view of one embodiment of the flushing assembly 402. The flushing assembly 402 includes a pump 510, a reservoir 512, and a controller 514. The controller 514 is coupleable, or coupled to, the pump 510, as illustrated by arrow 516. The pump 510 is coupled to the reservoir 512. A connector 518 in fluid communication with the reservoir 512 is configured and arranged to couple with the catheter 102, as illustrated by arrow 520. When the connector 518 is coupled to the catheter 102, acoustically-favorable medium disposed in the reservoir 512 can be output to the lumen 304 of the catheter 102, via the connector 518.

The connector 518 can be coupled to the catheter 102, via the main flush port 210. The connector 518 can, optionally, include a check valve to maintain a one-way flow of the acoustically-favorable medium into the lumen 304 of the catheter 102. Pumping acoustically-favorable medium into the lumen 304 of the catheter 102 may cause displacement of air bubbles disposed within the lumen 304. In some cases, the displaced air bubbles can be pushed out the output port 316 defined in a distal end of the catheter 102.

The connector 516 can be any connector suitable for forming a seal with the catheter 102 including, for example, a luer connector. The pump 510 can be any pump (e.g., a syringe pump, or the like) suitable for pumping acoustically-favorable media from the reservoir 512 to the catheter 102, at a desired pressure. The reservoir 512 can be of any shape and size suitable for supplying a sufficient volume of the acoustically-favorable medium to flush air bubbles from the lumen 304 of the catheter 102 one or more times.

The reservoir 512 can be coupled to the catheter 102 via tubing or, in some cases, a network of tubing. FIG. 5B is a schematic view of another embodiment of the flushing system 402 that includes tubing 530 coupleable, or coupled to, the reservoir 512. The tubing 530 can be either flexible for non-flexible and be any suitable bore or length for coupling the reservoir 512 to the catheter 102, and for pumping the acoustically-favorable medium at the desired pressure, or within the desired pressure range. In some cases, the connector 518 may be disposed on the tubing 530 for facilitating coupling with the catheter 102.

FIG. 5C is a schematic view of another embodiment of the flushing system 402, where the controller 514 is coupled to the processor 106, or the drive unit 110, or both, as shown by arrow 540. The controller 514 also includes a feedback loop system 542 that receives data from the processor 106 and uses the received data to either initiate a flushing procedure, or make adjustments to one or more flushing settings, or both.

In some instances, the feedback loop system 542 can be used to initiate a flushing procedure based, at least in part, on detected changes in the received data. For example, the controller 514 can be configured and arranged to detect changes in a signal-to-noise ratio of processed images from the data received from the processor 106. A decrease in signal-to-noise ratio can potentially be caused by air bubbles in the catheter lumen. Thus, a decrease in signal-to-noise ratio may be indicative of a degradation of the quality of generated images.

The controller 514 can be configured and arranged such that, when the signal-to-noise ratio falls below a predetermined level, the controller 514 initiates a flushing procedure. Additionally or alternately, the controller 514 can be configured and arranged such that, when the signal-to-noise ratio is reduced by a certain amount from an initial value, the controller 514 initiates a flushing procedure. For example, a flushing procedure can be initiated when the signal-to-noise ratio is reduced by a percentage amount including, for example, 1%, 5%, 10%, 15%, 20%, 25%, 50%, 75%, 100%, or more. It will be understood that a flushing procedure can be initiated when the signal-to-noise ratio is reduced by other percentage amounts.

In some instances, the feedback loop system 542 can be used to make adjustments to one or more flushing settings based, at least in part, on detected changes in the received data. The detected changes may include, for example, a reduction in the signal-to-noise ratio, as described above.

The controllable flushing settings may include, for example, the pressure of the acoustically-favorable medium input to the catheter lumen, the volume of the acoustically-favorable medium input to the catheter lumen, or the motor speed of the drive unit 110. Such adjustments to flushing settings can be implemented via connections between the controller 514 and the pump 510 and the drive unit 110. The controller 514 can be configured and arranged to simultaneously control operation of the pump 510 and the drive unit 110. In some cases, the controller 420 can be adapted to control operation of the drive unit 110 to retract the imaging core 306 prior to flushing the catheter lumen, or to cease rotation of the imaging core prior to flushing the catheter lumen, or both.

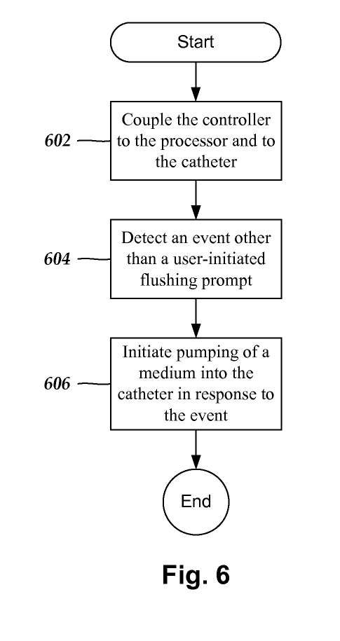

FIG. 6 is a flow diagram of one exemplary embodiment of a technique for flushing the lumen 304 of the catheter 102. In step 602, the controller 514 is coupled to both the catheter 102 and the processor 106. In step 604, the controller 514 detects an event other than a user-initiated flushing prompt. In step 606, the controller 514 initiates pumping of an acoustically-favorable medium into the lumen 304 of the catheter 102 in response to the event. Optionally, the controller 514 can adjust one or more flushing settings in response to the event. In some cases, the controller 514 may be coupled to the drive unit 110. The controller 514 may additionally be configured and arranged to initiate a pumping of an acoustically-favorable medium into the lumen 304 of the catheter 102 in response to a user-initiated flushing prompt.

It will be understood that each block of the block diagram illustrations, and combinations of blocks in the block diagram illustrations, as well any portion of the systems and methods disclosed herein, can be implemented by computer program instructions. These program instructions may be provided to a processor to produce a machine, such that the instructions, which execute on the processor, create means for implementing the actions specified in the block diagram block or blocks or described for the systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated, without departing from the scope or spirit of the invention.

The computer program instructions can be stored on any suitable computer-readable medium including, but not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computing device.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. An intravascular ultrasound imaging system comprising:
a catheter insertable into a patient blood vessel, the catheter having a distal end, a proximal end, a longitudinal axis, and defining a sealable lumen configured and arranged to receive an acoustically-favorable medium;
at least one transducer configured and arranged for insertion into the lumen of the catheter, the at least one transducer configured and arranged for transmitting acoustic signals, receiving reflected echo signals corresponding to the transmitted acoustic signals, and transforming the received echo signals to electrical signals;
a control module coupled to the at least one transducer, the control module comprising
a processor configured and arranged for processing electrical signals received from the at least one transducer, and
a drive unit configured and arranged for rotating the at least one transducer about, and along, the longitudinal axis of the catheter; and
a flushing assembly in fluid communication with the catheter lumen, the flushing assembly configured and arranged for flushing air bubbles formed in the acoustically-favorable medium disposed in the catheter lumen in response to an event other than a user-initiated flushing prompt, the flushing assembly comprising
a reservoir configured and arranged for containing acoustically-favorable medium for being input to the catheter lumen,
a pump coupled to the reservoir, the pump configured and arranged for pumping at least some of the acoustically-favorable medium from the reservoir to the catheter lumen,
a connector in fluid communication with the reservoir, the connector coupleable with the catheter lumen, and
a controller coupled to the pump, the controller configured and arranged for controlling operation of the pump.

2. The intravascular ultrasound imaging system of claim 1, wherein the control module further comprises a display coupled to the processor, wherein the display is configured and arranged to display images generated from the processed electrical signals received from the at least one transducer.

3. The intravascular ultrasound imaging system of claim 1, wherein the catheter defines an output port in fluid communication with the sealable lumen, the output port configured and arranged for flushing out gas from the sealable lumen.

4. The intravascular ultrasound imaging system of claim 1, wherein the flushing assembly is also configured and arranged to flush the lumen of the catheter in response to a user-initiated flushing prompt.

5. The intravascular ultrasound imaging system of claim 1, wherein the processor is coupled to the controller.

6. The intravascular ultrasound imaging system of claim 5, wherein the controller includes a feedback loop system for controlling at least one of the pressure within which at least some of the acoustically-favorable medium is input to the catheter lumen from the reservoir, or the volume of the acoustically-favorable medium input to the catheter lumen from the reservoir based, at least in part, on data received from the processor.

7. The intravascular ultrasound imaging system of claim 5, wherein the controller is coupled to the drive unit.

8. The intravascular ultrasound imaging system of claim 7, wherein the controller is configured and arranged to prohibit initiation of a flushing procedure unless the at least one transducer is in a retracted position within the lumen of the catheter.

9. The intravascular ultrasound imaging system of claim 1, wherein the pump is a syringe pump.

10. The intravascular ultrasound imaging system of claim 1, wherein the connector is disposed on tubing, the tubing in fluid communication with the reservoir.

11. A method for flushing a lumen of a catheter, the method comprising:
providing the intravascular ultrasound (IVUS) imaging system of claim 1;
coupling the processor of the IVUS imaging system to the controller of the flushing assembly;
receiving data, by the controller, from the processor of the IVUS imaging system, the data generated from electrical signals received from the at least one transducer of the IVUS imaging system as the at least one transducer rotates and moves along the lumen of the catheter of the IVUS imaging system;
detecting, by the controller, an event other than a user-initiated flushing prompt; and
initiating, by the controller, pumping of an acoustically-favorable medium from the reservoir of the IVUS imaging system to the lumen of the catheter in response to the event.

12. The method of claim 11, wherein detecting, by the controller, an event other than a user-initiated flushing prompt comprises detecting a degradation in image quality of images generated by the processor.

13. The method of claim 12, wherein detecting a degradation in image quality of images generated by the processor comprises detecting a reduction in signal-to-noise ratio.

14. The method of claim 13, wherein detecting a reduction in signal-to-noise ratio comprises detecting a reduction in signal-to-noise ratio to a level that is equal to or below a predetermined signal-to-noise ratio threshold level.

15. The method of claim 13, wherein detecting a reduction in signal-to-noise ratio comprises detecting a reduction in signal-to-noise ratio of at least a predetermined signal-to-noise ratio percentage.

16. The method of claim 11, wherein detecting, by the controller, an event other than a user-initiated flushing prompt comprises detecting an initiation of an imaging procedure.

17. The method of claim 11, wherein detecting, by the controller, an event other than a user-initiated flushing prompt comprises detecting a passing of a predetermined amount of time.

18. The method of claim 11, wherein initiating, by the controller, pumping of an acoustically-favorable medium from the reservoir of the IVUS imaging system to the lumen of the catheter in response to the event comprises controlling at least one of the flush pressure or the flush volume of the acoustically-favorable medium pumped into the lumen of the catheter.

19. The method of claim 11, further comprising initiating, by the controller, pumping of the acoustically-favorable medium from the reservoir of the IVUS imaging system to the lumen of the catheter in response to a user-initiated flushing prompt.

20. A computer-readable medium having processor-executable instructions, the processor-executable instructions when installed on a device enable the device to perform actions, comprising
receiving data from a processor of an intravascular ultrasound (IVUS) imaging system, the data generated from electrical signals received from at least one transducer of the IVUS imaging system as the at least one transducer rotates and moves along a lumen of a catheter of the IVUS imaging system;
detecting an event other than a user-initiated flushing prompt; and
pumping of an acoustically-favorable medium from a reservoir external to the catheter to the lumen of the catheter in response to the event.

21. A system for flushing a lumen of a catheter within which a rotatable intravascular ultrasound (IVUS) imager is disposed, comprising:
a processor, coupleable to the IVUS imager, for receiving data from the IVUS imager; and
a flushing-assembly controller, coupleable to the processor, for executing processor-readable instructions that enable actions, including:
receiving data from the processor, the data obtained as the IVUS imager rotates and moves along the lumen of the catheter;
detecting an event other than a user-initiated flushing prompt; and
pumping an acoustically-favorable medium from a reservoir external to the catheter to the lumen of the catheter in response to the event.
